(19) United States
(12) Patent Application Publication
(10) Pub. No.: US 2001/0028895 A1
(43) Pub. Date: Oct. 11, 2001

(54) Methods of treating alzheimer's disease

(76) Inventors: Charles L. Bisgaier, Ann Arbor, MI (US); Roger S. Newton, Ann Arbor, MI (US)

Correspondence Address: Patrea L Pabst Arnall Golden & Gregory LLP, 2800 One Atlantic Center, 1201 West Peachtree Street, Atlanta, GA, 30309-3450, US

(21) Appl. No.: 09/776,536

(22) Filed: Feb. 2, 2001

Related U.S. Application Data
(60) Provisional application No. 60/180,406, filed on Feb. 4, 2000.

(51) Int. Cl. A61K 9/127; A61K 35/78; A61K 38/44; A61K 38/17

(52) U.S. Cl. 424/450; 424/94.4; 514/21; 424/738; 424/754; 514/351

(57) ABSTRACT

Blood cholesterol levels are correlated with production of amyloid β protein (Aβ), and are predictors of populations at risk of developing AD. Methods for increasing HDL-cholesterol levels, HDL-apoA-I levels, or HDL function, can be used to decrease production of Aβ, thereby decreasing the risk of developing AD. Compounds which function as HDL include synthetic HDL which contains lipid such as phosphotidyl choline, phosphatidyl serine, phosphatidyl ethanolamine, and other phospholipids. Compounds which enhance HDL function include HDL associated proteins such as apo A1 or variants thereof including apo AI-Milano and biologically active peptides derived therefrom, reverse lipid transport (RLT) peptides, apoE, enzymes associated with HDL such as paraoxonase, and LCAT, alone or, more preferably, formulated in combination with liposomes or emulsions. These compositions can also be administered with compounds that increase HDL levels specifically, and thereby improve the HDL cholesterol to total cholesterol ratio or the apoA-I to total cholesterol ratio, and/or with compositions which are effective to improve the HDL or apoA-I to total blood cholesterol levels. Alternatively, or in addition, cholesteryl ester transfer protein inhibitors (CETP inhibitors) can be administered to the patients to treat or prevent Alzheimer's.

This application claims priority to U.S. Ser. No. 60/180,406 filed Feb. 4, 2000.

### BACKGROUND OF THE INVENTION

Alzheimer's disease (AD) is the most common cause of dementia in the aged population. The accumulation of large numbers of senile plaques containing the 40-42 amino acid amyloid β protein (Aβ) is a classic pathological feature of AD. Both genetic and cell biological findings suggest that the accumulation of Aβ in the brain is the likely cause of AD (Yankner, B. A. (1996) Neuron 16, 921-932 (1996); Selkoe, D. J. Science 275, 630-631 (1997)). Strong genetic evidence in support of the pathogenic role of Aβ came from the observation that individuals who inherit mutations in the amyloid precursor protein almost invariably develop AD at an early age. These mutations increase the production of a long variant of the Aβ peptide that forms senile plaques in the brain (Goate et al., Nature 349, 704-706 (1991)). Mutations and allelic variations in other genes that cause AD, including the presenilins and apolipoprotein E, also result in increased production or deposition of the Aβ peptide. Reiman, et al. (1996) N.E.J.Med. 334, 752-758, reported that in middle age, early to mid 50's, individuals who are homozygous for the apo E4 gene have reduced glucose metabolism in the same regions of the brain as in patients with Alzheimer's disease. These findings suggest that the pathological changes in the brain associated with this gene start early. Furthermore, individuals with Down's syndrome overexpress the amyloid precursor protein, develop Aβ deposits in the brain at an early age, and develop Alzheimer's disease at an early age. Finally, the Aβ protein has been demonstrated to be highly toxic to nerve cells. Thus, it is widely believed that drugs which decrease the levels of Aβ in the brain would prevent Alzheimer's disease.

Kuo, et al., Biochem. Biophys. Res. Comm. 252, 711-715 (1998) reported that based on postmortem data, there is a statistically significant correlation between high LDL cholesterol, Apo B, alpha-beta N-40, and alpha-beta N-42 and Alzheimer's Disease, independent of Apo E geneotype, indicating that elevated serum cholesterol, especially in the form of LDL, influences the expression of AD-related pathology. PCT US 99/06396 (WO 99/48488 published Sep. 30, 1999) by Childrens Medical Center Corporation and PCT/US98/25495 (WO 99/38498 published Aug. 5, 1999) by Warner-Lambert Company both describe administration of cholesterol lowering agents to treat or prevent Alzheimer's Disease. WO 99/38498 describes administration of plasma triglyceride level lowering agents, plasma cholesterol level lowering agents, or combinations thereof, to treat or prevent Alzheimer's disease.

It is an object of the present invention to provide pharmaceuticals to decrease the production of amyloid β protein (Aβ), and thereby to prevent or reduce the likelihood of developing AD.

It is a further object of the present invention to provide pharmaceutical treatments to treat AD in patients' having the neuropsychiatric or diagnostic criteria for AD.

### SUMMARY OF THE INVENTION

Blood cholesterol levels are correlated with production of amyloid β protein (Aβ), and are predictors of populations at risk of developing AD. Methods for increasing HDL-cholesterol levels, HDL-apoA-I levels, or HDL function, can be used to decrease production of Aβ, thereby decreasing the risk of developing AD. Compounds which function as HDL include synthetic HDL which contains lipids such as sphingomyelin, phosphotidyl choline, phosphatidyl serine, phosphatidyl ethanolamine, and other phospholipids, alone or in combination. Compounds which enhance HDL function include HDL associated proteins such as apo A1 or variants thereof including apo A1-Milano and biologically active peptides derived therefrom, reverse lipid transport (RLT) peptides, apoE, enzymes associated with HDL such as paraoxonase, and LCAT, alone or, more preferably, formulated in combination with liposomes or emulsions. The liposomes, alone or in combination with the HDL function enhancing proteins, act as a shuttle for the cholesterol from the cells to the liposomes. These compositions can also be administered with compounds that increase HDL levels specifically (i.e., not as a byproduct of decreasing LDL), and thereby improve the HDL cholesterol to total cholesterol ratio or the apoA-I to total cholesterol ratio, and/or with compositions which are effective to improve the HDL or apoA-I to total blood cholesterol levels. Alternatively, or in addition, cholesteryl ester transfer protein inhibitors (CETP inhibitors) can be administered to the patients.

Preferred populations to be treated include individuals with at least one allele for apo E4, high cholesterol, or a combination of at least one allelle for apoE4 and high cholesterol, defined as a blood cholesterol level of greater than 200 mg/dl, post menopausal women with high cholesterol levels—especially those who are not taking estrogen, or individuals which high blood cholesterol levels who are not obese are all at risk of developing AD if blood cholesterol levels are not decreased. In the preferred embodiment, individuals with these risk factors are treated to raise functional HDL levels prior to developing any mental impairment attributable to AD, based on accepted neuropsychiatric and diagnostic criteria in clinical practice.

### DETAILED DESCRIPTION OF THE INVENTION

Compositions to Decrease Production of Aβ.

Administration of synthetic HDL or compounds that enhance HDL can be used to decrease production of Aβ, thereby decreasing the risk of developing AD, have been developed. The same methods can also be used to treat patients who have already been diagnosed with AD. The synthetic HDL or compounds which enhance HDL function can also be administered with compounds which increase HDL cholesterol or apoA-I levels, such as CETP inhibitors. These can also be administered in combination with agents which lower LDL levels, for example, HMG CoA reductase inhibitors or compounds, such as intestinal cholesterol absorption inhibitors (e.g. beta-sitosterol, acylCoA:cholesterol acyltransferase (ACAT) inhibitors, saponins), bile acid sequestrants, fibrates, or niacin (nicotinic acid).

Synthetic HDL

Compositions which function as HDL, thereby effectively increasing HDL blood levels, include liposomal formulations as described in WO 95/23592 by the University of British Columbia. Preferably these are formed of phospholipids, such as sphingomyelin, phosphatidyl choline, phosphatidyl serine, and phosphatidyl ethanolamine, alone or in combination.

A preferred size of the liposomes is about 125 nm±50 nm (i.e., large unilamellar liposomes), although larger and smaller liposomes may also be useful.

Methods for making liposomes are well known, for example, as described in Chapter 1, Preparation of liposomes, in Liposome Drug Delivery Systems, Betageri, et al., (Technomic Publishing Co. 1993). These can include small unilamellar vesicles, large unilamellar vesicles, and multilamellar vesicles. The basic constituent typically is a phospholipid derived from natural and/or synthetic sources. Typically the main phospholipid will be phosphatidyl choline, but other neutral and charged lipids can be included. The traditional method of producing liposomes is to dissolve the constituent phospholipids in an organic solvent such as chloroform. The mixture can be filtered to remove insoluble matter and the solvent then removed under conditions of temperature and pressure that result in the formation of a dry lipid film. This film is then hydrated using an aqueous medium that can contain hydrophilic compounds, such as proteins and peptides. The hydration process can be controlled to control the resultant liposomes. When hydration occurs with mixing (for example, with hand shaking), multilamellar liposomes normally result. Smaller liposomes can be produced by the use of sonication and high pressure homogenization. Liposomes can also be filtered to prepare a more homogenous size preparation.

Emulsions are also prepared using standard processes, for example, by homogenization using a microfluidizer (Microfluidic Corporation) or an ultrasonic probe (Soniprobe). These can be characterized by laser diffractometer and/or photon correlation spectroscopy.

Compositions which Increase HDL Function.

Compositions which enhance HDL function include apo AI or variants thereof including Apo AI-Milano and biologically active amphipathic peptides derived therefrom, alone or in combination with liposomes or emulsions, for examples, as described in U.S. Pat. No. 5,876,968, and references cited therein, the teachings of which are incorporated herein.

Suitable apo A and apo A variant compositions are described in EP 0469017 by Pharmacia Upjohn, EP 067703 by Farmatolia, and U.S. Patent No. 5,834,596 to Ageland, et al. Proapolipoprotein AI is described in U.S. Pat. No. 5,059,528 to Bollen, et al. Synthetic amphipathic peptides are described in PCT/US00/8788 by Dasseaux, et al. Peptide/lipid complexes are described in PCT/US98/20330 by Dasseaux. Either compounds are described in PCT/US00/8799 by Esperion Therapeutics.

Human apolipoprotein A-I (apo A-I) possesses multiple tandem repeating 22-meramphipathic alpha-helixes. Computer analysis and studies of model synthetic peptides and recombinant protein-lipid complexes of phospholipids have suggested that apo A-I interacts with HDL surface lipids through cooperation among its individual amphipathic helical domains. Each of the eight tandem repeating 22-mer domains of apo A-I: residues 44-65, 66-87, 99-120, 121-142, 143-164, 165-186, 187-208, and 220-241 were synthesized. Among the 22-mers, only the N- and C-terminal peptides (44-65 and 220-241) were effective in clarifying multilamellar vesicles (MLVs) of dimyristoylphosphatidylcholine (DMPC). These two peptides also exhibited the highest partition coefficient into 1-palmitoyl-2-oleoyl-sn-glycero-3-phosphatidylcholine liposomes, the highest exclusion pressure for penetration into an egg yolk phosphatidylcholine monolayer, and the greatest reduction in the enthalpy of the gel-to-liquid crystalline phase transition of DMPC MLVs. These results suggest that the strong, lipid-associating properties of apo A-I are localized to the N- and C-terminal amphipathic domains. Peptides containing only one (18A) or two (37pA) amphipathic helical segments stimulate as much cholesterol efflux from both mouse macrophages and L-cells as apo AI. Acceptor efficiency is dependent on the number of amphipathic helical segments per molecule. When the helical content of 18A is increased by neutralizing the charges at the ends of the peptide (Ac-18A-NH2), there is a substantial increase in the efficiency for cholesterol efflux (EC50 18A=17 micrograms/mL vs Ac-18A-NH2=6 micrograms/mL). The efficiency with which the peptides stimulated cholesterol efflux is in order of their lipid affinity), and this order is similar for phospholipid efflux. Dimeric amphipathic helical peptides compete for high-affinity HDL binding sites on cholesterol-loaded fibroblasts and display saturable high-affinity binding to the cell surface. In contrast, peptides with a single helix have little or no ability to remove cellular cholesterol and phospholipid, or to interact with HDL binding sites, suggesting that cooperativity between two or more helical repeats is required for these activities. Thus, synthetic peptides comprising dimers of a structural motif common to exchangeable apolipoproteins can mimic apolipoprotein A-I in both binding to putative cell-surface receptors and clearing cholesterol from cells.

Trimeric apolipoprotein (apo)AI(145-183) peptides composed each of two amphipathic alpha-helical segments, are branched onto a covalent core matrix and the construct recombined with phospholipids. The complexes generated with the trimeric-apoAI(145-183) bind specifically to HeLa cells with comparable affinity to the DMPC apoAI complexes; they are a good competitor for binding of apoAI to both HeLa cells and Fu5AH rat hepatoma cells; and promote cholesterol efflux from Fu5AH cells with an efficiency comparable with the apo AI/lipid complexes. These peptides are described by Palgunachari, et al., Arterioscler Thromb Vasc Biol. 16:328-338 (1996); Yancey, et al., Biochemistry. 34:7955-7965 (1995); Mendez, et al., J Clin Invest. 94:1698-1705 (1994); and Nion, et al., Atherosclerosis. 141:227-235 (1998).

Plasma Cholesterol Level Lowering Agents and Plasma Triglyceride Level Lowering Agents

These compositions can be administered in combination with plasma cholesterol level lowering agents and plasma triglyceride level lowering agents such as HMG CoA reductase inhibitors, bile acid sequestrants, agents that block intestinal cholesterol absorption, saponins, neomycin, and acyl CoA:cholesterol acyl transferase inhibitors.

Representative HMG CoA reductase inhibitors include the statins, including lovastatin, simvastatin, compactin, fluvastatin, atorvastatin, cerivastatin, and pravastin. Representative fibrates include clofibrate, fenofibrate, gemfibrozil, or bezafibrate. Compounds which inhibit cholesterol biosynthetic enzymes, including 2,3-oxidosqualene cyclase, squalene synthase, and 7-dehydrocholesterol reductase, can also be used. Representative compositions which decrease uptake of dietary cholesterol include the bile acid binding resins (cholestryramine and colestipol). Probucol, nicotinic acid, garlic and garlic derivatives, and psyllium are also used to lower blood cholesterol levels. Probucol and the fibrates increase the metabolism of cholesterolcontaining lipoproteins.

Plasma triglyceride lowering agents also include niacin, carboyxalkylethers, thiazolinediones, eicosapentaenoic acid, EPA, and acylCoA:cholesteryl acyltransferase (ACAT).

Cholesteryl Ester Transfer Protein (CETP) Inhibitors

Patients can also be treated with CETP inhibitors, alone or in combination with the compositions which act as HDL or act to enhance HDL function. Representative compounds include PD 140195 as described by Bisgaier, et al., LIPIDS 29(12), 811-818 (1994); tetrahydroquinoline derivatives described in EPA 987251 by Pfizer, pyridine derivatives described in DE 19731609-C3 by Searle & Co.; triazole derivates described in WO 99/14204 by Searle & Co; substituted tetrahydro-napthalene derivates described in DE 741050 by Bayer AG; benzyl-biphenyl derivatives described in DE 741400 by Bayer AG; tetrahydro-quinoline derivatives described by Bayer AG phenylamine derivatives described by JP 11049743 by Japan Tobacco Inc.; erabulenols described by Tomoda, et al., J. Antibiotics 51(7), 618-623 (1998); BM99-1 and BM99-2 described by JP09059155 by Kaken Pharm Co Ltd.; tetracyclic catechols as described by Xia,et al., 212^{th} Amer. Chem. Soc. Nat. Meeting, Orlando, Fla. Aug. 25-29, 1996; and vaccines, described in WO 99/20302 by Rittershaus; Rittershaus, et al., Arterioscler. Thromb. Vasc. Biol. 20:2106-2112 (2000); WO 99/15655 by Monsanto; and WO 9741227 by T Cell Science. Antisense is described in DE 19731609 by Boehringer Ingelheim Pharm KG.

Methods of Treatment

The compositions are typically administered orally, in tablet form, once daily, using the same or lower dosages as are currently used to treat atherosclerosis. Lower dosages would more typically be used when the treatment is prophylactic. As noted above, some compositions, such as the liposomes, and emulsions of compounds enhancing HDL function, will more typically be administered by means of injection.

Compositions are administered in an amount and for a length of time effective to increase relative HDL to total cholesterol levels sufficient to decrease deposition of plaque in the brains of patients at risk of developing Alzheimers. The increase can be due to the administration of the “synthetic” HDL or to enhancement of function of the endogenous HDL.

Individuals at increased risk for Aβ accumulation and Alzheimer's disease are those who carry a copy of the apolipoprotein E4 gene (Strittmatter et al., (1993) Proc. Natl. Acad. Sci. U.S.A. 90, 1977-1981), those with trisomy 21 (Down's syndrome) (Mann and Esiri, (1989) J. Neurol. Sci. 89, 169-179)), and individuals who carry a mutation in one of the genes that encode the amyloid precursor protein, presenilin-1 or presenilin-2 (reviewed in Yankner, 1996). In addition, individuals with a family history of Alzheimer's disease have been documented to be at increased risk of Alzheimer's disease (Farrer et al., (1989) Ann. Neurol. 25, 485-492; van Duijn et al., (1991) Int. J. Epidemiol. 20 (suppl 2), S13-S20), and could therefore benefit from prophylactic treatment.

Several risk factors for developing AD have been identified by others.

These include:

Individuals with apo E4 and high cholesterol, defined as a blood cholesterol level of greater than 200 mg/dl,

Post menopausal women with high cholesterol, especially those who are not taking estrogen,

Young individuals with high blood cholesterol levels who are not obese (age 48-65 yrs),

Individuals with high blood cholesterol levels who have a family history of AD, and

All adult individuals with Down's syndrome.

These individuals are all at risk of developing AD. In the preferred embodiment, individuals with these risk factors are treated to raise the HDL functional levels prior to developing any mental impairment attributable to AD using accepted neuropsychiatric and diagnostic criteria for probable Alzheimer's disease (McKhahn et al. (1984) Neurology 34:939-944).

Individuals can be screened using standard blood tests for cholesterol, apoE4, and/or total lipoprotein levels, as well as by taking a medical and family history. Importantly, these individuals should also be screened for their HDL-cholesterol or apoA-I levels. Individuals with low HDL-cholesterol or apo A-I levels can particularly benefit from the treatment described herein.

In the preferred embodiment, compositions are administered in the following ranges:

HDL (protein) up to 100 mg/kg body weight, preferred 5-75 mg/kg, most preferably around 30-60 mg/kg.

RLT (protein) are administered up to 100 mg/kg body weight, preferably 1-50 mg/kg, most preferably 5-30 mg/kg.

Liposomes are administered up to 500 mg/kg body weight, preferably 25-300 mg/kg, most preferably 75-250 mg/kg.

The compositions can be administered in a single or multiple dosages. For multiple administration, the compositions for IV infusion are given usually once a week, however they may be given every two to four days up to once every year. An effective dose and treatment regimen is given to block the onset of AD or to treat AD and can be assessed by periodic evaluations of the patient. Clinical diagnosis can be performed by interview with the subject and relatives with questionaire techniques familar to those skilled in the evaluation of conditions of dementia.

Modifications and variations of the methods and compositions described herein will be obvious to those skilled in the art from the foregoing detailed description and are intended to fall within the scope of the appended claims.

We claim:

1. A method for decreasing the production of Aβ comprising administering an effective amount of a composition selected from the group consisting of synthetic HDL compositions, compositions selectively enhancing HDL function with minimal effect on LDL levels, cholesteryl ester transfer protein inhibitors in a pharmaceutically acceptable vehicle, and combinations thereof, to a person with elevated cholesterol levels who is at risk of, or exhibits the symptoms of, Alzheimer's disease.

2. The method of claim 1 wherein the composition is a synthetic HDL composition.

3. The method of claim 2 wherein the synthetic HDL composition comprises liposomes.

4. The method of claim 1 wherein the composition is a composition selectively enhancing HDL function.

5. The method of claim 4 wherein the composition comprises apo AI or a variant or polypeptide derived therefrom.

6. The method of claim 5 wherein the variant is apo AI Milano

7. The method of claim 5 wherein the polypeptide is an amphipathic peptide that can act as an apolipoprotein and can act as a structural component of synthetic HDL.

8. The method of claim 1 wherein the composition is a cholesteryl ester transfer protein inhibitor.

9. The method of claim 1 further comprising administering a compound selected from the group consisting of plasma cholesterol level lowering agents and plasma triglyceride level lowering agents.

10. The method of claim 9 wherein the compound is selected from the group consisting of HMG CoA reductase inhibitors, bile acid sequestrants, agents that block intestinal cholesterol absorption, saponins, neomycin, and acyl CoA:cholesterol acystransferase inhibitors.

11. The method of claim 10 wherein the HMG CoA reductase inhibitor is selected from the group consisting of lovastatin, simvastatin, compactin, fluvastatin, atorvastatin, cerivastatin, and pravastin.

12. The method of claim 10 wherein the fibrate is selected from the group consisting of clofibrate, fenofibrate, gemfibrozil, and bezafibrate.

13. The method of claim 10 wherein the compound is selected from the group of compounds inhibiting cholesterol biosynthetic enzymes consisting of 2,3-oxidosqualene cyclase, squalene synthase, and 7-dehydrocholesterol reductase.

14. The method of claim 10 wherein the compound is selected from the group consisting of compounds decreasing uptake of dietary cholesterol, bile acid binding resins, probucol, nicotinic acid, garlic and garlic derivatives, and psyllium.

15. The method of claim 10 wherein the compound is selected from the group consisting of niacin, carboyxalkylethers, thiazolinediones, eicosapentaenoic acid, EPA, and acyl-CoA:cholesteryl acyltransferase (ACAT).

16. The method of claim 1 wherein the person is at risk of developing Alzheimer's disease but does not display neurologic deficiencies and an effective amount of the composition is administered to decrease deposition of alpha-beta plaque.

17. The method of claim 16 wherein the person carries the apolipoprotein E4 gene.

18. The method of claim 16 wherein the person has trisomy 21 (Down's syndrome).

19. The method of claim 16 wherein the person carries one or more mutations in the genes that encode amyloid β protein, amyloid precursor protein, presenilin-1 or presenilin-2.

20. The method of claim 16 wherein the person has a family history of Alzheimer's disease or dementing illness.

21. The method of claim 16 wherein the person is a post menopausal woman with high cholesterol.

22. The method of claim 16 wherein the person has high blood cholesterol levels who is not obese.

23. The method of claim 1 wherein the person has Alzheimer's disease and an effective amount of composition is administered to slow or decrease deposition of alpha-beta plaque in the person's brain.
